SEQUENCE LISTING (10 sequences)

SEQ ID NO: 1 - protein, 37 aa, Homo sapiens
HDEFERHAEGTFTSDVSSYLEGQAAKEFIAWLVKGRG

SEQ ID NO: 2 - protein, 31 aa, Homo sapiens
HAEGTFTSDVSSYLEGQAAKEFIAWLVKGRG

SEQ ID NO: 3 - protein, 30 aa, Homo sapiens
HAEGTFTSDVSSYLEGQAAKEFIAWLVKGR

SEQ ID NO: 4 - protein, 39 aa, Gila monster
HGEGTFTSDLSKQMEEEAVRLFIEWLKNGGPSSGAPPPS

SEQ ID NO: 5 - protein, 31 aa, Artificial sequence
  Synthetic peptide
  C-16 (20)..(20): C-16 fatty acid (palmitic acid) attached with a glutamic acid spacer on the lysine residue at position 26 of the peptide precursor
HAEGTFTSDVSSYLEGQAAKEFIAWLVRGRG

SEQ ID NO: 6 - protein, 44 aa, Artificial sequence
  Synthetic peptide
  Lixisenatide (38)..(38): derived from the first 39 amino acids in the sequence of the peptide exendin-4, omitting proline at position 38 and adding six lysine residues KKKKKK
HGEGTFTSDLSKQMEEEAVRLFIEWLKNGGPSSGAPPSKKKKKK

SEQ ID NO: 7 - protein, 645 aa, Artificial Sequence
  Synthetic protein
  albiglutide (60)..(60): GLP-1 dimer fused to human albumin.  The two GLP-1-likes domains have a single amino acid substitution relative to GLP-1(7-36)
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 275 aa, Artificial Sequence
  Synthetic protein
  Dulaglutide (28)..(28): GLP-1 analogue covalently linked to a human IgG4-Fc heavy chain by a small peptide linker
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 31 aa, Artificial Sequence
  Synthetic peptide
  semaglutide (2)..(2): Xaa = alpha-aminoisobutyric acid
  semaglutide (20)..(20): Lys20 is acylated with C-18 stearic diacid (AEEAc-AEEAc-?-Glu-17-carboxyheptadecanoyl)
HXEGTFTSDVSSYLEGQAAKEFIAWLVRGRG

SEQ ID NO: 10 - protein, 30 aa, Artificial Sequence
  Synthetic peptide
  Taspoglutide (2)..(2): Xaa = 2-methylalanine
  Taspoglutide (29)..(29): Xaa = 2-methylalanine
  Taspoglutide (30)..(30): Xaa = L-argininamide
HXEGTFTSDVSSYLEGQAAKEFIAWLVKXX

(19) United States
(12) Patent Application Publication
(10) Pub. No.: US 2021/0162013 A1
(43) Pub. Date: Jun. 3, 2021

(54) COMPOSITIONS AND METHODS FOR IMPROVING THE BIOAVAILABILITY OF GLP1 AND ANALOGUES THEREOF

(71) Applicants: Brown University, Providence, RI (US); Therapyx, Inc., Buffalo, NY (US)

(72) Inventors: Edith Mathiowitz, Providence, RI (US); Nejat Egilmez, Louisville, KY (US); Thomas Conway, Hamburg, NY (US)

(21) Appl. No.: 17/265,747

(22) PCT Filed: Aug. 5, 2019
(86) PCT No.: PCT/US2019/045145

§ 371 Date: Feb. 3, 2021

Related U.S. Application Data
(60) Provisional application No. 62/714,454, filed on Aug. 3, 2018.

(51) Int. Cl. A61K 38/26; A61K 47/59; A61K 47/12; A61K 9/00; A61P 3/10

(52) U.S. Cl. CPC A61K 38/26; A61K 47/593; A61P 3/10; A61K 9/0053; A61K 47/12

(57) ABSTRACT

Compositions including Glucagon-like peptide-1 (GLP-1) or an analogue thereof such as exenatide, liraglutide, lixisenatide, albiglutide, dulaglutide, semaglutide, or taspoglutide, entrapped in or incorporated into a polymeric particles are provided. Typically, the particles are composed of one or more biodegradable polyesters or polyanhydrides, or a combination thereof, for example as copolymers or a blend to two or more polymers or copolymers. In some embodiments, the particles do not include a poly(lactide-co-glycolide). The particles can be microparticles or nanoparticles. In some embodiments, the particles are formed by Phase Inversion Nanoencapsulation (PIN). Pharmaceutical compositions and dosage forms, including formulations suitable for oral delivery, are also provided. Method of treating subject in need thereof, for example subjects with diabetes, by administering the subject an effect amount of the disclosed compositions, are also provided.

### CROSS-REFERENCE TO RELATED APPLICATION

This application claims benefit and priority to U.S. Application No. 62/714,454, filed Aug. 3, 2018, the disclosure of which is incorporated herein by reference.

### REFERENCE TO THE SEQUENCE LISTING

The Sequence Listing submitted as a text file named “BU_2535_PCT” created on Aug. 5, 2019, and having a size of 12,676 bytes is hereby incorporated by reference pursuant to 37 C.F.R. § 1.52(e)(5).

### FIELD OF THE INVENTION

The field of the invention is generally in the field of drug delivery, particularly oral delivery of bioactivity agents.

### BACKGROUND OF THE INVENTION

Abnormalities of the incretin axis are connected to the pathogenesis of type 2 diabetes mellitus. Glucagon-like peptide-1 (GLP-1) and gastroinhibitory intestinal peptide constitutes >90% of all the incretin function, and increases in GLP-1 can improve beta cell health in a glucose-dependent manner (post-prandial hyperglycemia) and suppress glucagon (fasting hyperglycemia). Native GLP-1 has a very short plasma half-life. GLP-1 analogues typically correct this drawback without eliminating the bioactivity of GLP-1, thus enhancing the viability of GLP-1 receptor modulation as a treatment for diabetes.

Although GLP-1 and GLP-1 analogues are highly biologically active following intravenous or subcutaneous injection, a non-injectable alternative for activating GLP-1 receptor signaling is desirable.

Further, improving the absorption of peptides such as GLP-1 so as to minimize the actual amount of peptide required in various buccal or enteral formulations remains a major challenge.

Thus, it is an object of the invention to provide improved compositions for delivering GLP-1 and analogues thereof and improved methods for delivering GLP-1 and analogues thereof.

### SUMMARY OF THE INVENTION

Compositions including Glucagon-like peptide-1 (GLP-1) or an analogue thereof entrapped in or incorporated into polymeric particles are provided. The compositions typically increase the bioavailability, bioactivity, or a combination thereof of GLP-1 or a GLP-1 analogue when administered to a subject in need thereof relative to administering the subject GLP-1 or the GLP-1 analogue alone.

The particles can be composed of one or more biodegradable polyesters or polyanhydrides, or a combination thereof. In some embodiments, the particles are formed of a copolymer of two or more polymers, or a blend of two or more polymers, copolymers, or combination thereof. The particles can include, or be formed of, poly(adipic acid) (PAA) or lactic acid units, for example poly-L-lactic acid, poly-D-lactic acid, poly-D,L-lactic acid, poly-L-lactide, poly-D-lactide, or poly-D,L-lactide, (PLA), poly(lactide-co-glycolide) (PLGA), or a copolymer or blend thereof.

Optionally, the particles have a core of a first polymer and a coating or shell of a second polymer. For example, the particles can be in the form of double-walled microspheres. Double-walled microspheres can have a core formed from PLA or PLGA and a coating formed from one or more polyanhydrides.

In some embodiments, the particles do not contain as the sole polymer carboxyl terminated poly(lactide-co-glycolide). Carboxyl terminated poly(lactide-co-glycolide) refers to PLGA that contains a carboxyl group at one or both termini of the polymer. The carboxyl group can be in its protonated state (PLGA-COOH) or salt state (PLGA-COO⁻, i.e., ionic state). The salt state can be formed, for example, via a reaction between the carboxyl group in its protonated form and a base.

The particles can be microparticles or nanoparticles. In some embodiments, the particles are formed by phase inversion nanoencapsulation (PIN).

Pharmaceutical compositions including the disclosed particles and a pharmaceutically acceptable carrier are also provided. For example, formulations suitable for oral delivery are disclosed.

Methods of treatment are also provided. For example, a method of treating a subject in need thereof can include administering to the subject an effective amount of particles loaded with GLP-1 and/or an analogue thereof to reduce fasting blood glucose, post-prandial blood glucose, glycated haemoglobin (HbA1c), weight, daily insulin requirements, or a combination thereof. Thus, in some embodiments, the composition is administered to a subject with diabetes in an effective amount to reduce one or more symptoms of the diabetes.

Methods of improving the cardiovascular condition and enhancing neuroprotection in a subject are also provided. A method of improving the cardiovascular condition in a subject can include, for example, administering to the subject in need thereof an effective amount of the composition to increase myocardial contractility, hypertension, the thickness of the endothelium, lipid profile, or a combination thereof. A method of enhancing neuroprotection can include administering to a subject in need thereof an effective amount of the composition to improve cognition, memory, spatial learning, or a combination thereof in the subject.

Any of the disclosed methods can include oral delivery of the composition.

### BRIEF DESCRIPTION OF THE DRAWINGS

FIGS. 1A-1C are bar graphs showing the in vitro release profiles of total GLP-1 (7-36 plus 9-36 amide) release from PAA (FIG. 1A), PLA (FIG. 1B), and PLGA (FIG. 1C) nanoparticles (10 mg) following hydration with 0.2 ml media. Cumulative release was determined at the indicated times (hours) in triplicate. All formulations had a GLP-1 loading of 2.5%.

FIG. 2 is a bar graph showing the stability profile of GLP-1 encapsulated in PAA, PLA, and PLGA particles. Cumulative total and bioactive GLP-1 released from each formulation at 3 hours is shown as percent of theoretical maximum peptide loading. Each sample was analyzed in triplicate. Error bars=standard deviation. The difference between PAA total and bioactive peptide was significant (p=0.0004). The differences for PLA and PLGA were not significant (p>0.066).

FIGS. 3A-3C are line graphs showing the in vivo efficacy in an intraperitoneal glucose tolerance test (IPGTT). BKS.Cg WT mice received 10 mg of GLP-1-loaded particles (250 μg of total peptide) immediately prior to glucose injection. Control mice received blank particles mixed with soluble peptide. Serum samples were collected just prior to glucose injection (shown as time 0) and at the indicated times following glucose injection. Each time point is an average of 4 mice. Error bars=standard deviation. The differences between time 0 and later time points were significant in mice treated with PAA at 15 and 45 min (p<0.015) and for PLA at 45 and 60 min (p<0.001). The differences were not significant for PLGA. Glucose levels in treated mice are shown as % of glucose levels in control animals.

FIG. 4 is a line graph showing PIN/PLA GLP-1 bioavailability as measured by serum glucose (mg/dL) over time (minutes post-treatment). Each point represents the average of 4 mice. Error bars=standard deviation. The differences between glucose only and PIN/PLA GLP-1 were significant at 10, 20 and 30 minutes (p<0.05).

FIG. 5 is a line graph showing the short-term serum pharmacokinetics of PIN/PLA GLP-1. Each point represents average serum GLP-1 values from 3 mice. Error bars=standard error. All experimental time points were significantly different from controls (p<0.011).

FIG. 6 is a line graph showing the long-term serum pharmacokinetics of PIN/PLA GLP-1. Each point represents average serum GLP-1 values from 3 mice. Error bars=standard error. All experimental time points were significantly different from controls (p≤0.01).

FIG. 7 is a line graph showing the effect of oral PIN/PLA GLP-1 administration on blood glucose levels in Lepr^{db/db} mice. The glucose readings for both the PIN/PLA GLP-1 and soluble peptide groups were normalized to control group and are expressed as % of control value for each time point. Each data point is representative of 4 (control and soluble peptide groups) or 8 mice (PIN/PLA GLP-1). Error bars=standard deviation. (*) Represents a statistically significant difference of p≤0.005.

### DETAILED DESCRIPTION OF THE INVENTION

### I. Compositions

The compositions contain olymeric particles, which contain an active agent such as GLP-1 (7-36 amide) or an analogue thereof encapsulated therein. The particles are suitable for delivery in vivo of a biologically active form of the active agent.

The particles typically increase the bioavailability or bioactivity of the active agent in vivo relative to the bioavailability of a control. Bioavailability can be determined by, for example, the serum level or serum half-life of the active agent following in vivo delivery. For example, an increase in the bioavailability can be demonstrated by an increase in serum concentration of the active agent over time or at a discrete time point relative to a control.

In some embodiments, the compositions have improved bioactivity compared to a control. Bioactivity can be measured as, for example, a down stream pharmacological, physiological, or biochemical response to the active agent compared to a control. For example when the active agent can control serum glucose level, the bioavailability can be measured as the ability of the composition to lower glucose levels compared to a control.

The compositions described herein are suitable for oral administration and the bioavailability of the GLP-1 or analogue thereof following oral administration is at least 40%, at least 45%, or at least 50% compared to the bioavailability of a control formulation that contains the same dosage of GLP-1 or the analogue thereof in the absence of the particles and where the control formulation is administered intraperitoneally. The bioavailability can be determined using an intraperitoneal glucose tolerance test (IPGTT) and comparing serum glucose levels over time for the orally administered composition with serum glucose levels over time for an intraperitoneally injected formulation containing unencapsulated GLP-1 or the analogue thereof at same dosage as the composition, such as described in Example 2.

The bioavailability and bioactivity of an active agent in the compositions can be compared to the same active agent delivered in the absence of particles, or via an alternative delivery system such as, for example, particles with the same or a similar size distribution that are formed from PLGA-COOH as the only polymer.

A. Particles

The particles can be microparticles or nanoparticles, or a combination thereof.

The microparticles can be microspheres, microcapsules, and/or structures that may not be readily placed into either of the above two categories, all with mean particle sizes of less than about 1000 microns. The microparticles can have a mean particle size ranging from about 1 micron or greater up to about 1000 microns, such as from 1 micron to 100 microns, from 1 micron to 500 microns, from 2 μm to 10 μm, from 10 microns to 50 microns, from 50 microns to 100 microns, from 100 microns to 500 microns, or from 500 microns to 1000 microns. The microparticles may be microspheres that are substantially spherical colloidal structures formed from biocompatible polymers having a size ranging from about 1 micron or greater up to about 1000 microns, such as from 1 micron to 100 microns, from 1 micron to 500 microns, from 10 microns to 50 microns, from 50 microns to 100 microns, from 100 microns to 500 microns, or from 500 microns to 1000 microns. The microparticles may be microcapsules, which contain a core and shell.

Nanoparticles have a mean particle size of less than one micron, and include nanospheres and nanocapsules. In certain embodiments, the nanoparticles have a mean particle size of about 500 nm, 200 nm, 100 nm, 50 nm, 10 nm, or 1 nm. The nanoparticles can have a mean particle size in a range from 1 micron to 100 microns, from 1 micron to 500 microns, from 10 microns to 50 microns, from 50 microns to 100 microns, from 100 microns to 500 microns, or from 500 microns to 1000 microns.

The particles may contain nanoparticles and microparticles. Optionally, the particles have a mean particle size ranging from 1 nm to 1000 μm, from 100 nm to 1000 μm, from 0.5 μm to 50 μm, from 2 μm to 10 μm, or from 0.5 μm to 2 μm.

Mean particle size generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. The diameter of a non-spherical particle may refer to the largest linear distance between two points on the surface of the particle. Mean particle size can be measured using methods known in the art, such as dynamic light scattering.

A composition containing microparticles and/or nanoparticles may include particles of a range of particle sizes. The particle size distribution may be uniform, e.g., within less than about a 20% standard deviation of the mean volume or mean particle size or within about 10% of the median volume or mean particle size.

The compositions can be polydisperse or monodisperse. Monodisperse size distribution describes a population of nanoparticles or microparticles where all of the particles are the same or nearly the same size. A monodisperse size distribution refers to particle distributions in which 90% of the population or particles lies within 15% of the median particle size, or within 10% of the median particle size, or within 5% of the median particle size.

The particles may be of any suitable size or range of sizes for the desired delivery method. Generally, the diameter of the particles ranges from 1 nm to 1000 μm, from 100 nm to 1000 μm, or from 1 to 1000 μm.

In some embodiments, the mean diameter of the particles in the formulation is in the range of 0.5 μm to 50 μm, 2 μm to 10 μm, or 0.5 μm to 2 μm. The mean diameter of the particles in the formulation can be in the range of 2 μm to 10 μm for delivery of the bioactive agent locally to the gastrointestinal tract, or in the range of 0.5 μm to 2 μm for delivery of the bioactive agent systemically.

The bioactive agent can be released from the particles for at least 12 hours, at least 24 hours, at least 36 hours, at least 48 hours, up to 100 hours, up to 200 hours, or up to 300 hours or longer following administration.

B. Bioactive Agents

The particles typically include a bioactive agent. The bioactive agent is typically Glucagon-like peptide-1 (GLP-1) or a truncated biologically active portion thereof or an analogue thereof.

1. Glucagon-Like Peptide-1

Glucagon-like peptide-1 (GLP-1), a member of the glucagon peptide family, is a 30 amino acid long peptide hormone deriving from the tissue-specific posttranslational processing of the proglucagon gene.

Human GLP-1 (1-37) has the amino acid sequence:

(SEQ ID NO: 1)
HDEFERHAEGTFTSDVSSYLEGQAAKEFIAWLVKGRG.

The initial product GLP-1 (1-37) is susceptible to amidation and proteolytic cleavage, which gives rise to the two truncated and equipotent biologically active forms, GLP-1 (7-36) amide and GLP-1 (7-37).

Human GLP-1 (7-37) has the amino acid sequence:

(SEQ ID NO: 2)
HAEGTFTSDVSSYLEGQAAKEFIAWLVKGRG.

Human GLP-1 (7-36) has the amino acid sequence:

(SEQ ID NO: 3)
HAEGTFTSDVSSYLEGQAAKEFIAWLVKGR

Active GLP-1 contains two a-helices from amino acid position 13-20 and 24-35 (of SEQ ID NO:1) separated by a linker region.

DPP-IV cleaves the peptide bond in Ala8-Glu9 (of SEQ ID NO:1), and the resulting metabolite GLP-1(9-36)-NH₂ is found to have 100-fold lower binding affinity compared to the intact peptide (Manadhar and Ahn, J. Med. Chem. 2015, 58, 1020-1037). The metabolite also exhibits negligible agonistic activity (>10000-fold decrease).

Orally administered GLP-1 in a dose-escalating schedule (doses of 0.5, 1.0, 2.0, and 4.0 mg) was reported to (i) induce a rapid and dose-dependent increase in plasma drug concentrations; (ii) induce a potent effect on insulin release; and (iii) suppressed ghrelin secretion (Beglinger, et al., Clin Pharmacol Ther. 2008 October; 84(4):468-74). However, Beglinger reported bioavailabilities lower than 10%, with a mean absolute bioavailability of 4%, relative to intravenous administration of GLP-1. Further, native GLP-1 has a very short plasma half-life and is generally not suitable for therapeutic use except by continuous infusion. For example, it is possible to normalize or improve the glycemic control in type 2 diabetic patients by both intravenous and subcutaneous infusion of GLP-1 at doses of ˜4 ng·kg⁻¹·min⁻¹ or higher, however, these studies ranged from 4 to 6 hours in duration for either fasting patients or patients receiving a single meal (Larsen and Hylleberg, Diabetes Care 2001 August; 24(8): 1416-1421). Continuous 48-hour subcutaneous infusion of GLP-1 at a rate of ˜4-8 ng·kg⁻¹·min⁻¹ also lowered fasting and postprandial glucose values in type 2 diabetic patients, and another study showed that fasting serum glucose decreased by 76.2, 53.9, 37.0 and 22.7 mg/dl for the 8.5, 5.0, 2.5 and 1.25 pmol/kg/min rGLP-1 groups, respectively, compared to a decrease of 1.1 mg/dl for placebo (Torekov., et al., Diabetes Obes Metab., 2011 July; 13(7):639-43).

2. Glucagon-Like Peptide-1 Analogues

Modifying the two sites in the GLP-1 molecule susceptible to cleavage: the position 8 alanine and the position 34 lysine, can help prolong the half-life of GLP-1. These, and other chemical modifications, help in creating compounds known as GLP-1 receptor agonists, which have a longer half-life, and can be used for therapeutic purposes.

Suitable GLP-1 analogues include, for example, exenatide (BYETTA®, BYDUREON®), liraglutide (VICTOZA®, SAXENDA®), lixisenatide (LYXUMIA®, ADLYXIN®), albiglutide (TANZEUM™), dulaglutide (TRULICITY®), semaglutide (OZEMPIC®), and taspoglutide.

a. Exenatide

Exenatide, a functional analogue of GLP-1, is a synthetic version of exendin-4, a hormone found in the saliva of the Gila monster. Exenatide has the amino acid sequence:

(SEQ ID NO: 4)
HGEGTFTSDLSKQMEEEAVRLFIEWLKNGGPSSGAPPPS.

BYETTA® is an immediate-release exenatide formulated for subcutaneous (SC) injection. The recommended dosage for treating type 2 diabetes mellitus is 5 μg SC every 12 hours within 60 minutes prior to meal initially; after 1 month, may increased to 10 μg every 12 hours. BYDUREON® BCISE™ is an extended-release exenatide formulated for subcutaneous (SC) injection. The recommended dosage for treating type 2 diabetes mellitus is 2 mg subcutaneously once every 7 days (weekly), administered any time of day, with or without meals.

b. Liraglutide

Liraglutide is a long-acting, fatty acylated GLP-1 analogue with prolonged action and half-life of 11-15 hours. The improved properties of liraglutide are credited to the attachment of the fatty acid palmitic acid to GLP-1 that reversibly binds to albumin and protects it from degradation and elimination and facilitates slow and consistent release. Liraglutide has the amino acid sequence HAEGTFTSDVSSYLEGQAAKEFIAWLVRGRG (SEQ ID NO:5), and has a C-16 fatty acid (palmitic acid) attached with a glutamic acid spacer on the lysine residue at position 26 of the peptide precursor (bold/italics in SEQ ID NO:5). Liraglutide is 97% homologous to native human GLP-1 with a substituted arginine for lysine at position 34.

VICTOZA® and SAXENDA® are liraglutide formulations for subcutaneous injection. A recommended dose for VICTOZA for treating type 2 diabetes mellitus is 0.6 mg SC every day for 1 week initially, then increase to 1.2 mg or 1.8 mg every day based on clinical response.

c. Lixisenatide

Lixisenatide is “des-38-proline-exendin-4 (Heloderma suspectum)-(1-39)-peptidylpenta-L-lysyl-L-lysinamide,” meaning it is derived from the first 39 amino acids in the sequence of the peptide exendin-4, omitting proline at position 38 and adding six lysine residues. The amino acid sequence of lixisenatide is

(SEQ ID NO: 6)
HGEGTFTSDLSKQMEEEAVRLFIEWLKNGGPSSGAPPSKKKKKK.

ADLYXIN® and LYXUMIA® are lixisenatide formulations for subcutaneous injection. The initial recommended dose for treating type 2 diabetes mellitus is 10 μg everyday for 14 days, followed by 20 μg everyday beginning on day 15.

d. Albiglutide

Albiglutide is a dipeptidyl peptidase-4-resistant GLP-1 dimer fused to human albumin. The two GLP-1-likes domains have a single amino acid substitution relative to GLP-1(7-36). The amino acid sequence for albiglutide is:

(SEQ ID NO: 7)
HGEGTFTSDVSSYLEGQAAKEFIAWLVKGRHGEGTFTSDVSSYLEGQ
AAKEFIAWLVKGRDAHKSEVAHRFKDLGEENFKALVLIAFAQYLQQ
CPFEDHVKLVNEVTEFAKTCVADESAENCDKSLHTLFGDKLCTVAT
LRETYGEMADCCAKQEPERNECFLQHKDDNPNLPRLVRPEVDVMCT
AFHDNEETFLKKYLYEIARRHPYFYAPELLFFAKRYKAAFTECCQAA
DKAACLLPKLDELRDEGKASSAKQRLKCASLQKFGERAFKAWAVA
RLSQRFPKAEFAEVSKLVTDLTKVHTECCHGDLLECADDRADLAKYI
CENQDSISSKLKECCEKPLLEKSHCIAEVENDEMPADLPSLAADFVES
KDVCKNYAEAKDVFLGMFLYEYARRHPDYSVVLLLRLAKTYETTLE
KCCAAADPHECYAKVPDEFKPLVEEPQNLIKQNCELFEQLGEYKFQN
ALLVRYTKKVPQVSTPTLVEVSRNLGKVGSKCCKHPEAKRMPCAED
YLSVVLNQLCVLHEKTPVSDRVTKCCTESLVNRRPCFSALEVDETYV
PKEFNAETFTFHADICTLSEKERQIKKQTALVELVKHKPKATKEQLK
AVMDDFAAFVEKCCKADDKETCFAEEGKKLVAASQAALGL.

TANZEUM™ is an albiglutide formulation for subcutaneous injection. The initial recommended dose for treating type 2 diabetes mellitus is 30 mg SC once weekly, which may be increased to 50 mg once weekly if the glycemic response is inadequate.

e. Dulaglutide

Dulaglutide is GLP-1 receptor agonist that includes a dipeptidyl peptidase-IV-protected GLP-1 analogue covalently linked to a human IgG4-Fc heavy chain by a small peptide linker. The amino acid sequence for dulaglutide is:

(SEQ ID NO: 8)
HGEGTFTSDVSSYLEEQAAKEFIAWLVKGGGGGGGSGGGGSGGGGS
AESKYGPPCPPCPAPEAAGGPSVFLFPPKPKDTLMISRTPEVTCVVVD
VSQEDPEVQFNWYVDGVEVHNAKTKPREEQFNSTYRVVSVLTVLH
QDWLNGKEYKCKVSNKGLPSSIEKTISKAKGQPREPQVYTLPPSQEE
MTKNQVSLTCLVKGFYPSDIAVEWESNGQPENNYKTTPPVLDSDGSF
FLYSRLTVDKSRWQEGNVFSCSVMHEALHNHYTQKSLSLSLG.

TRULICITY is a dulaglutide formulation for subcutaneous injection. The initial recommended dose for treating type 2 diabetes mellitus is once-weekly SC injection 0.75 mg, which may be increased to 1.5 mg once weekly for additional glycemic control.

f. Semaglutide

Semaglutide is GLP-1 analogue that differs to others in the following ways amino acid substitutions at position 8 (alanine to alpha-aminoisobutyric acid, a synthetic amino acid) and position 34 (lysine to arginine), and acylation of the peptide backbone with a spacer and C-18 fatty di-acid chain to lysine at position 26. These changes permit a high-affinity albumin binding and stabilize semaglutide against dipeptidylpeptidase-4, giving it a long plasma half-life. The amino acid sequence for semaglutide is: HXEGTFTSDVSSYLEGQAAKEFIAWLVRGRG (SEQ ID NO:9), where X is alpha-aminoisobutyric acid and Lys20 is acylated with C-18 stearic diacid (AEEAc-AEEAc-γ-Glu-17-carboxyheptadecanoyl).

OZEMPIC® is a semaglutide formulation for subcutaneous injection. The initial recommended dose for treating type 2 diabetes mellitus is 0.25 mg SC every week for 4 weeks, then increase the dosage to 0.5 mg weekly.

g. Taspoglutide

Taspoglutide is the 8-(2-methylalanine)-35-(2-methylalanine)-36-L-argininamide derivative of the amino acid sequence 7-36 of human GLP-1. Thus, the sequence of taspoglutide is HXEGTFTSDVSSYLEGQAAKEFIAWLVKXX (SEQ ID NO:10), wherein X2 is 2-methylalanine, X29 is 2-methylalanine, and X30 is L-argininamide.

Studies show that 20 mg taspoglutide administered once weekly by subcutaneous injection for 4 weeks, followed by dose maintenance at 20 mg, or titration to 30 mg (20/30) or 40 mg (20/40) once weekly for an additional 4 weeks was safe, well tolerated at high doses and efficacious for lowering HbA(1c) (Ratner, et al., Diabet Med. 2010 May; 27(5):556-62. doi: 10.1111/j.1464-5491.2010.02990.x).

C. Polymers

The disclosed particles include core particles and core-shell particles. Thus, in some embodiments, the particles have a shell, and in some embodiments they do not have a shell. In some embodiments, the particles have a coating, such as for example, a bioadhesive coating.

1. Core Polymer

a. Type of Polymer

Polymers for forming the polymeric particles are also provided.

Biodegradable polymers can be used as the core polymer for drug delivery applications, wherein one or more encapsulated active agents are released over time as the core polymer degrades.

In some embodiments the particle contains one or more biodegradable polyesters (e.g., polyhydroxyesters), or one or more polyanhydrides, or blends or copolymers thereof. In some embodiments, such as poly(lactic acid), poly(glycolic acid), and poly(lactic-co-glycolic acid).

The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(ε-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof.

Copolymers including lactic acid and glycolic acid units, such as various forms of poly(lactic acid-co-glycolic acid) and poly(lactide-co-glycolide) can be characterized by the ratio of lactic acid:glycolic acid, collectively referred to herein as “PLGA”; and polyacrylates, and derivatives thereof. In some embodiments, the particles do not solely contain carboxyl terminated poly(lactide-co-glycolide). However, the particles may contain a blend of polymers, wherein one of the polymers in the blend is PLGA or carboxyl terminated PLGA.

The polyanhydrides can be formed from the polymerization of dicarboxylic acids. The dicarboxylic acids can be linear saturated dicarboxylic acids or linear unsaturated dicarboxylic acids. Suitable polyanhydrides include polyadipic anhydride, polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios. For example, a copolymer could contain a first polyanhydride and a second polyanhydride at molar ratios ranging from 5:95 to 95:5, 20:80 to 80:20; or 30:70 to 70:30.

Exemplary polymers also include copolymers of polyethylene glycol (PEG) and the aforementioned polyesters, such as various forms of PLGA-PEG, PGA-PEG, or PLA-PEG copolymers, collectively referred to herein as “PEGylated polymers”. In certain embodiments, the PEG region can be covalently associated with polymer to yield “PEGylated polymers” by a cleavable linker.

In some embodiments, the polymer does not have PEG conjugated thereto. In some embodiments, the polymer does not have a terminal carboxylic acid, or corresponding salt thereof. The particles can contain one or a mixture of two or more polymers. The polymers may be used alone, as physical mixtures (blends), or as co-polymers. The particles may contain other entities such as stabilizers, surfactants, or lipids.

In specific embodiments, the particles are formed of PAA (poly-adipic acid) or PLA (poly-lactic acid).

In vitro and in vivo studies disclosed in the Examples show that GLP-1 nanoparticles produced using PLA as the base polymer provided superior in vitro characteristics and in vivo efficacy. In vitro release assays showed that PLA nanoparticles released active GLP-1 peptide over an eight-hour period and IPGTT studies in BKS.Cg WT mice demonstrated that GLP-1 PLA nanoparticles exhibited the longest lasting efficacy of the three different preparations. Further characterization of the PLA GLP-1 formulation formed by phase inversion nanoencapsulation (PIN/PLA GLP-1) demonstrated a relative bioavailability of >43% and that a single oral dose resulted in physiologically-relevant levels of serum GLP-1 for at least 8 hours. Further, therapeutic studies in BKS.Cg Lepr^{db/db} diabetic mice showed that the PIN/PLA GLP-1 formulation was also effective in reducing blood glucose levels in diabetic mice.

Optionally, the core comprises one or more bioadhesive polymers or is coated with one or more bioadhesive polymers. Suitable bioadhesive polymers are described in the Shell Polymer section below.

2. Size of Polymer

The weight average molecular weight range for the core polymer in a core polymer solution can range from about 1 kDa to about 200 kDa, or from about 2 kDa to about 200 kDa, or from about 2 kDa to about 150 kDa, or from about 2 kDa to about 100 kDa, or from about 1 kDa to about 25 kDa, or from about 1.5 kDa to about 15 kDa, or from about 2 kDa to about 12 kDa, or from about 2 kDa to about 11 kDa. In some embodiments one or more polymers, or all of the polymers, forming the particles are about 1 kDa, 2, kDa, 3 kDa, 4 kDa, 5 kDa, 6 kDa, 7 kDa, 8 kDa, 9 kDa, 10 kDa, 11 kDa, or 12 kDa.

Suitable core polymer concentrations in the core polymer solution range from about 0.01 to about 50% (weight/volume), depending primarily upon the molecular weight of the core polymer and the resulting viscosity of the core polymer solution. In general, the low molecular weight polymers permit usage of higher polymer concentrations. The preferred concentration range is from about 0.1% to about 10% (weight/volume), optionally the concentration is about 5% (weight/volume) or lower. Polymer concentrations ranging from about 1% to about 5% (weight/volume) are particularly useful for the methods described herein.

The viscosity of the core polymer solution preferably is less than about 3.5 centipoise and more preferably less than about 2 centipoise, although higher viscosities such as about 4 or even about 6 centipoise are possible depending upon adjustment of other parameters such as molecular weight.

It will be appreciated by those of ordinary skill in the art that polymer concentration, polymer molecular weight and viscosity are interrelated, and that varying one will likely affect the others.

3. Shell Polymer

The particles may contain a second polymer that forms the shell of the particles. The second polymer can be any of the polymers described above with respect to the core polymer. However, the second polymer is different than the core polymer. In one embodiment, the second polymer is a biodegradable, non-bioadhesive polymer. In other embodiments, the shell polymer is a non-degradable, non-bioadhesive polymer. In still other embodiments, the shell polymer is a bioadhesive polymer, for example a biodegradable, bioadhesive polymer.

In double-walled nanoparticles, the second polymer is the shell polymer. However, in nanoparticles containing three or more polymers, the second polymer is not the shell polymer. Rather the shell polymer is the final polymer added to form the multi-walled nanoparticles.

In particles containing more than three walls, a third, fourth, etc. polymer are included in forming the particles. Like the second polymer, these subsequently added polymers polymer can be any of the polymers described above with respect to the core polymer. However, they are different polymers than the core polymer and the polymers in the preceding layers.

For drug delivery applications, the shell polymer is preferably a biodegradable polymer, such as those described above for the core polymer. The shell polymer can prevent burst release of the agent by preventing release of the agent that is on the surface of the core. In some embodiments the shell polymer is bioadhesive. Alternatively, the outermost wall of the multi-walled nanoparticles may contain a bioadhesive coating or a matrix coating.

Triple-walled particles containing a biodegradable core and a second biodegradable polymer layer can further contain a bioadhesive shell which adheres the particles to the mucosa at the desired site of release in the gastrointestinal tract. Alternatively, double-walled particles containing a biodegradable core and a biodegradable shell can be dispersed in a bioadhesives matrix. Suitable bioadhesives materials are described below.

The molecular weight range for the shell polymer typically ranges from about 1 kDa to about 150 kDa, such as from about greater than 1 kDa to about 100 kDa, from about 2kDa to about 50 kDa, or from 10 kDa to 50 kDa.

Excipients may also be added to the shell polymer to alter its porosity and permeability. Suitable excipients may include inorganic and organic materials such as sucrose, hydroxypropyl cellulose, sodium chloride, sodium chloride, xylitol, sorbitol, lactose, dextrose, maltodextrins and dextrates

Excipients may also be added to the shell polymer to alter its hydration and disintegration properties. Suitable pH dependent enteric excipients may include cellulose acetate phthalate, acdisol, hydroxypropyl cellulose, and hydroxypropyl methyl cellulose.

a. Bioadhesive Polymers

Particularly preferred polymers for the shell polymer are bioadhesive polymers. A bioadhesive polymer is one that binds to mucosal epithelium under normal physiological conditions. Bioadhesion in the gastrointestinal tract proceeds in two stages: (1) viscoelastic deformation at the point of contact of the synthetic material into the mucus substrate, and (2) formation of bonds between the adhesive synthetic material and the mucus or the epithelial cells. In general, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., van der Waals interactions) and stronger specific interactions, which include hydrogen bonds. The hydrophilic functional groups primarily responsible for forming hydrogen bonds are the hydroxyl and the carboxylic groups.

Bioadhesives with varying hydration times and durations of bioadhesiveness in aqueous media could directly impact the performance of oral formulations. Bioadhesives have demonstrated the ability to promote intimate contact with the GI mucosa for prolonged periods of time leading to increased bioavailability of small molecule drugs. Additionally, it has been reported that a relationship exists between increased bioadhesiveness and increased nanoparticle uptake. Given the therapeutic aims of the oral formulation, taking into account the pharmacokinetics of the release and mucus turnover, choosing a polymer that will remain bioadhesive for the desired duration is of great importance to the field of oral drug delivery.

For example, to achieve prolonged release in the intestines of a small molecule over the period of hours, a bioadhesive with a low rate of hydration might be ideal, e.g. poly(fumaric-co-sebacic anhydride). However, as a carrier to enhance nanoparticle uptake, the bioadhesive polymer may function to promote contact between the nanoparticle and the GI mucosa for a short time until the nanoparticle can achieve mucus permeation and then dissolve prior to nanoparticle uptake.

Representative bioadhesive polymers include bioerodible hydrogels, such as those described by Sawhney, et al., in Macromolecules, 1993, 26:581-587, the teachings of which are incorporated herein by reference. Other suitable bioadhesive polymers are described in U.S. Pat. No. 6,235,313 to Mathiowitz et al., the teachings of which are incorporated herein by reference, and include polyhydroxy acids, such as poly(lactic acid), polystyrene, polyhyaluronic acids, casein, gelatin, glutin, polyanhydrides, polyacrylic acid, alginate, chitosan; polyacrylates, such as poly(methyl methacrylates), poly(ethyl methacrylates), poly butylmethacrylate), poly(isobutylmethacrylate), poly(hexylmethacrylate), poly(isodecyl methacrylate), poly(lauryl methacrylate), poly(phenyl methacrylate), poly (methyl acrylate), poly(isopropyl acrylate), poly(isobutyl acrylate), and poly(octadecyl acrylate); polyacrylamides; poly(fumaric-co-sebacic)acid, poly(bis carboxy phenoxy propane-co-sebacic anhydride), polyorthoesters, and combinations thereof.

Suitable polyanhydrides include polyadipic anhydride, polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios.

Optionally, the shell polymer is a blend of hydrophilic polymers and bioadhesive hydrophobic polymers. Suitable hydrophilic polymers include, but are not limited to, hydroxypropylmethylcellulose, hydroxypropylcellulose, carboxymethylcellulose, polyvinylalcohols, polyvinylpyrollidones, and polyethylene glycols. The hydrophobic polymer may contain gastrosoluble polymers that dissolve in stomach contents, such as Eudragit® E100. The hydrophobic polymer may contain entero-soluble materials that dissolve in the intestine above pH 4.5, such as Eudragit® L-100, Eudragit® S-100, cellulose acetate phthalate, hydroxypropyl methylcellulose phthalate, hydroxypropyl methylcellulose acetate succinate, Eastacryl® 30D dispersion from Eastman Chemicals., Sureteric® (polyvinyl acetate phthalate) and Acryl Eze®.

In some embodiments, the bioadhesive material is a polymer containing a plurality of aromatic groups containing one or more hydroxyl groups. Such polymers are described in detail in U.S. Patent Application Publication No. 2005/0201974 to Schestopol, et al., the disclosure of which is incorporated herein by reference. Suitable aromatic moieties include, but are not limited to, catechol and derivatives thereof, trihydroxy aromatic compounds, or polyhydroxy aromatic moieties. In one embodiment, the aromatic moiety is 3,4-dihydroxyphenylalanine (DOPA), tyrosine, or phenylalanine, all of which contain a primary amine In a preferred embodiment, the aromatic compound is 3,4-dihydroxyphenylalanine.

The degree of substitution by the aromatic moiety can vary based on the desired adhesive strength; it may be as low as 10%, 20%, 25%, 50%, or up to 100% substitution. On average at least 50% of the monomers in the polymeric backbone are substituted with the at least one aromatic moiety. Preferably, 75-95% of the monomers in the backbone are substituted with at least one of the aromatic groups or a side chain containing one or more aromatic groups. In the preferred embodiment, on average 100% of the monomers in the polymeric backbone are substituted with at least one of the aromatic groups or a side chain containing one or more of the aromatic groups.

The bioadhesive polymer can be formed by first coupling the aromatic compound to a monomer or monomers and polymerizing the monomer or monomers to form the bioadhesive polymer. In this embodiment, the monomers may be polymerized to form any polymer, including biodegradable and non-biodegradable polymers. Alternatively, polymer backbones can be modified by covalently attaching the aromatic moieties to the polymer backbone. In those embodiments where the aromatic moieties are grafted to a polymer chain, the aromatic moieties can be part of a compound, side chain oligomer, and/or polymer.

Regardless of the mechanism, the monomer or polymer generally contains one or more reactive functional groups which can react with the aromatic moiety to form a covalent bond. In one embodiment, the aromatic moiety contains an amino group and the monomer or polymer contains one or more amino reactive groups. Suitable amino reactive groups include, but are not limited to, aldehydes, ketones, carboxylic acid derivatives, cyclic anhydrides, alkyl halides, acyl azides, isocyanates, isothiocyanates, and succinimidyl esters.

The polymer that forms that backbone of the bioadhesive material containing the aromatic groups may be any non-biodegradable or biodegradable polymer. In the preferred embodiment, the polymer is a hydrophobic polymer. In one embodiment, the polymer is a biodegradable polymer.

Suitable polymer backbones include, but are not limited to, polyanhydrides, polyamides, polycarbonates, polyalkylenes, polyalkylene oxides such as polyethylene glycol, polyalkylene terepthalates such as poly(ethylene terephthalate), polyvinyl alcohols, polyvinyl ethers, polyvinyl esters, polyethylene, polypropylene, poly(vinyl acetate), poly vinyl chloride, polystyrene, polyvinyl halides, polyvinylpyrrolidone, polyhydroxy acids, polysiloxanes, polyurethanes and copolymers thereof, modified celluloses, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, and polyacrylates such as poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate).

Examples of preferred biodegradable polymers for forming the shell polymer include synthetic polymers such as poly hydroxy acids, such as polymers of lactic acid and glycolic acid, polyanhydrides, poly(ortho)esters, polyesters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. In one embodiment, the shell polymer is a copolymer of maleic anhydride and butadiene containing DOPA, tyrosine, and/or phenyl alanine groups. In another embodiment, the polymer is a copolymer of maleic anhydride and ethylene containing DOPA, tyrosine, and/or phenyl alanine groups. Other suitable monomers that can be copolymerized with maleic anhydride include vinyl acetate and styrene.

The polymer may be also a known bioadhesive polymer that is hydrophilic or hydrophobic. Hydrophilic polymers include CARBOPOL™ (a high molecular weight, crosslinked, acrylic acid-based polymers manufactured by NOVEON™), polycarbophil, cellulose esters, and dextran.

In some embodiments, one can use non-biodegradable polymers, especially hydrophobic polymers. Examples of preferred non-biodegradable polymers include ethylene vinyl acetate, poly(meth) acrylic acid, copolymers of maleic anhydride with other unsaturated polymerizable monomers, poly(butadiene maleic anhydride), polyamides, copolymers and mixtures thereof, and dextran, cellulose and derivatives thereof.

b. Bioadhesive Oligomers

Shell polymers with enhanced bioadhesive properties can be provided wherein bioadhesive monomers or oligomers, such as anhydride monomers or oligomers, are incorporated into the polymer. The oligomer excipients can be blended or incorporated into a wide range of hydrophilic and hydrophobic polymers including proteins, polysaccharides and synthetic biocompatible polymers. Anhydride oligomers may be combined with metal oxide particles to improve bioadhesion even more than with the organic additives alone. The incorporation of oligomer compounds into a wide range of different polymers which are not normally bioadhesive can dramatically increases their adherence to tissue surfaces, such as mucosal membranes.

As used herein, the term “anhydride oligomer” refers to a diacid or polydiacids linked by anhydride bonds, and having carboxy end groups linked to a monoacid such as acetic acid by anhydride bonds. The anhydride oligomers have a molecular weight less than about 5000, typically between about 100 and 5000 Da, or are defined as including between one to about 20 diacid units linked by anhydride bonds. In one embodiment, the diacids are those normally found in the Krebs glycolysis cycle. The anhydride oligomer compounds have high chemical reactivity.

The oligomers can be formed in a reflux reaction of the diacid with excess acetic anhydride. The excess acetic anhydride is evaporated under vacuum, and the resulting oligomer, which is a mixture of species which include between about one to twenty diacid units linked by anhydride bonds, is purified by recrystallizing, for example from toluene or other organic solvents. The oligomer is collected by filtration, and washed, for example, in ethers. The reaction produces anhydride oligomers of mono and poly acids with terminal carboxylic acid groups linked to each other by anhydride linkages.

The anhydride oligomer may be hydrolytically labile. As analyzed by gel permeation chromatography, the molecular weight may be, for example, on the order of 200 to 400 for fumaric acid oligomer (FAPP) and 2000 to 4000 for sebacic acid oligomer (SAPP). The anhydride bonds can be detected by Fourier transform infrared spectroscopy by the characteristic double peak at 1750 cm 1 and 1820 cm 1, with a corresponding disappearance of the carboxylic acid peak normally at 1700 cm 1.

In one embodiment, the oligomers may be made from diacids described for example in U.S. Pat. No. 4,757,128 to Domb et al., U.S. Pat. No. 4,997,904 to Domb, and U.S. Pat. No. 5,175,235 to Domb et al., the disclosures of which are incorporated herein by reference. For example, monomers such as sebacic acid, bis(p carboxy phenoxy)propane, isophathalic acid, fumaric acid, maleic acid, adipic acid or dodecanedioic acid may be used.

c. Bioadhesive Additives

Additives can be added to the shell polymer to alter the properties of the shell polymer provided the additives do not adversely affect the formation of the nanoparticles. Suitable additives include, but are not limited to, dyes and excipients which alter porosity, permeability, hydration, and/or disintegration properties.

Organic dyes because of their electronic charge and hydrophobicity/hydrophilicity can be used to either increase or decrease the bioadhesive properties of polymers when incorporated into the shell polymer. Suitable dyes include, but are not limited to, acid fuchsin, alcian blue, alizarin red s, auramine o, azure a and b, Bismarck brown y, brilliant cresyl blue ald, brilliant green, carmine, cibacron blue 3GA, congo red, cresyl violet acetate, crystal violet, eosin b, eosin y, erythrosin b, fast green fcf, giemsa, hematoxylin, indigo carmine, Janus green b, Jenner's stain, malachite green oxalate, methyl blue, methylene blue, methyl green, methyl violet 2b, neutral red, Nile blue a, orange II, orange G, orcein, paraosaniline chloride, phloxine b, pyronin b and y, reactive blue 4 and 72, reactive brown 10, reactive green 5 and 19, reactive red 120, reactive yellow 2,3, 13 and 86, rose bengal, safranin o, Sudan III and IV, Sudan black B and toluidine blue.

The bioadhesives properties can also be improved by adding metal compounds, such as water-insoluble metal oxides and metal hydroxides, which are capable of becoming incorporated into and associated with a polymer to thereby improve the bioadhesiveness of the polymer as described in U.S. Pat. No. 5,985,312, which is incorporated herein by reference in its entirety. As defined herein, a water-insoluble metal compound is defined as a metal compound with little or no solubility in water, for example, less than about 0.0-0.9 mg/ml.

The water-insoluble metal compounds, such as metal oxides, can be incorporated by one of the following mechanisms: (a) physical mixtures which result in entrapment of the metal compound; (b) ionic interaction between metal compound and polymer; (c) surface modification of the polymers which would result in exposed metal compound on the surface; and (d) coating techniques such as fluidized bead, pan coating or any similar methods known to those skilled in the art, which produce a metal compound enriched layer on the surface of the device.

The water-insoluble metal compounds can be derived from metals including calcium, iron, copper, zinc, cadmium, zirconium and titanium. For example, a variety of water-insoluble metal oxide powders may be used to improve the bioadhesive properties of polymers such as ferric oxide, zinc oxide, titanium oxide, copper oxide, barium hydroxide, stannic oxide, aluminum oxide, nickel oxide, zirconium oxide and cadmium oxide. The incorporation of water-insoluble metal compounds such as ferric oxide, copper oxide and zinc oxide can tremendously improve adhesion of the polymer to tissue surfaces such as mucosal membranes, for example in the gastrointestinal system. The polymers incorporating a metal compound thus can be used to form or coat the particles to improve their bioadhesive properties.

4. Active Agent Loading

The bioactive agent is typically dispersed or encapsulated in the core polymer. The loading range for the agent within the nanoparticles is from about 0.01 to about 80% (agent weight/polymer weight), or from 0.01% to about 50% (wt/wt), or from about 0.01% to about 25% (wt/wt), or from about 0.01% to about 10% (wt/wt), or from about 0.1% to about 5% (wt/wt).

For large biomolecules, such as proteins and nucleic acids, typical loadings are from about 0.01% to about 5% (wt/wt), or from about 0.01% to about 2.5% (wt/wt), or from about 0.01% to about 1% (wt/wt).

D. Pharmaceutically Acceptable Carriers

The compositions may also include one or more pharmaceutically acceptable carriers, excipients or diluents. The pharmaceutical formulations may be produced using standard procedures. Pharmaceutically carriers, excipients or diluents for different dosage forms are known in the art, and described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995).

The disclosed compositions can be formulated with appropriate pharmaceutically acceptable carriers into pharmaceutical compositions for administration to an individual in need thereof. The formulations can be administered enterally (e.g., oral) or parenterally (e.g., by injection or infusion).

The disclosed compositions can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

1. Different Types of Excipients

Typical classes of carriers, excipients and/or diluents include, but are not limited to, buffers, surfactants, preservatives, binders, lubricants, disintegrators, swelling agents, fillers, stabilizers, and combinations thereof. The term “pharmaceutically acceptable excipient” also includes all components of any coating formed around the bioactive agent particles, which may include plasticizers, pigments, colorants, stabilizing agents, and glidants.

Excipients may also be included in the composition to alter its porosity and permeability. Suitable excipients may include inorganic and organic materials such as sucrose, hydroxypropyl cellulose, sodium chloride, sodium chloride, xylitol, sorbitol, lactose, dextrose, maltodextrins, and dextrates.

Excipients may also be included in the composition to alter its hydration and disintegration properties. Suitable pH dependent enteric excipients may include cellulose acetate phthalate.

Excipients may also be added as a “wicking agent” to regulate the hydration of the composition. Suitable excipients may include acdisol, hydroxypropyl cellulose, hydroxypropyl methyl cellulose, and cellulose acetate phthalate.

Polyadipic anhydride prevents coalescence of drug domains within a spray-dried product resulting in increased drug surface area available for dissolution. Additionally, adipic acid monomer generated during polymer degradation increases acidity in the microenvironment of a spray-dried drug particle. By changing the pH, some of the drugs may become more soluble.

a. Coating Materials

The composition may further be coated with a polymer to facilitate oral administration, and to protect the bioactive agent from acidic environments, such as found in the stomach. Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, methacrylic resins, zein, shellac, and polysaccharides.

Additionally, the coating material may contain conventional carriers such as plasticizers, pigments, colorants, glidants, stabilization agents, pore formers and surfactants.

b. Surfactants

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, Pluronics, stearoyl monoisopropanolamide, and polyoxyethylene hydrogenated tallow amide. Examples of amphoteric surfactants include sodium N-dodecyl-.beta.-alanine, sodium N-lauryl-beta-iminodipropionate, myristoamphoacetate, lauryl betaine lauryl sulfobetaine, and lecithin.

2. Liquid or Injectable Drug Delivery Dosage Forms

Preferably the composition is in the form of a plurality of particles, which are dispersed or suspended in a pharmaceutically acceptable carrier, such as a diluent. Generally the plurality of particles is dispersed or suspended in the carrier immediately before it is administered. Suitable diluents include an appropriate buffer, for example, phosphate buffered saline or other physiologically compatible solutions. Surfactant such as TWEEN™, or polyethylene glycol, sodium lauryl sulfate, sodium caprate, pluronics, Span 80 and lecithin may be incorporated into the suspension or dispersion as needed.

In some embodiments, the disclosed compositions are administered systemically by, for example, injection or infusion. In some embodiments, the compositions are administered locally by injection or infusion. In more specific embodiments, the compositions are administered to the central nervous system, particularly the brain, by convection enhanced delivery (CED).

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required nanocarrier size in the case of dispersion and/or by the use of surfactants. In many cases, isotonic agents, for example, sugars or sodium chloride are included

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric, or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, exemplary methods of preparation include vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

3. Solid Drug Delivery Dosage Forms

Optionally, the composition can be further formulated into solid drug delivery dosage forms, such as tablets, capsules, multiparticulates, beads, or granules. The solid dosage forms may be prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose (HPMC), sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. Optional pharmaceutically acceptable excipients present in the tablets, multiparticulate formulations, beads, granules, or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet, multiparticulate, bead, or granule remains intact during storage and until administration. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

If desired, the tablets, beads, granules, or particles may also contain minor amounts of nontoxic auxiliary substances, such as wetting or emulsifying agents, dyes, pH buffering agents, and/or preservatives.

4. Enteral Dosage Forms

The compositions are preferably in a form suitable for enteral administration, preferably oral administration. Exemplary routes of enteral administration include, but are not limited to, sublingual, buccal, and oral. Suitable dosage forms for enteral administration include, but are not limited to, tablets, capsules, caplets, solutions, suspensions, syrups, powders, or thin films.

The enteral dosage forms can contain one or more excipients including any number of medically or pharmaceutically acceptable excipients such as preservatives, lipids, fatty acids, waxes, surfactants, plasticizers, porosigens, antioxidants, bulking agents, buffering agents, chelating agents, cosolvents, water-soluble agents, insoluble agents, metal cations, anions, salts, osmotic agents, synthetic polymers, biological polymers, hydrophilic polymers, polysaccharides, sugars, hydrophobic polymers, hydrophilic block copolymers, hydrophobic block copolymers, block copolymers containing hydrophilic and hydrophobic blocks. Such excipients can be used singly or in combinations of two or more excipients when preparing particle compositions. These excipients can be useful in order to alter or affect drug release, water uptake, polymer degradation, stability of the bioactive agent, among other properties.

The one or more excipients can be incorporated during formation of the particles, for example by addition to one or more of the polymer solutions. Alternatively, the one or more excipients can be combined with the particles after they are formed, when the particles are formulated into pharmaceutically acceptable compositions. The one or more excipients can be used at a concentration from about 1% to about 90% by weight of the composition.

Examples of water soluble and hydrophilic excipients include poly(vinyl pyrrolidone) or PVP and copolymers containing one or more blocks of PVP along with blocks of other biocompatible polymers (for example, poly(lactide) or poly(lactide-co-glycolide) or polycaprolactone); poly(ethylene glycol) or PEG and copolymers containing blocks of PEG along with blocks of other biocompatible polymers (for example, poly(lactide) or poly(lactide-co-glycolide) or polycaprolactone); poly(ethylene oxide) or PEO, and copolymers containing one or more blocks of PEO along with blocks of other biocompatible polymers (for example, poly(lactide) or poly(lactide-co-glycolide) or polycaprolactone) as well as block copolymers containing PEO and poly(propylene oxide) or PPO such as the triblock copolymers of PEO-PPO-PEO (such as Poloxamers™, Pluronics™); and, modified copolymers of PPO and PEO containing ethylene diamine (Poloxamines™ and Tetronics™).

The particles described herein can be formulated as a tablet, capsule, or caplet. The tablet, capsule or caplet can be coated with a modified release coating, such as an enteric coating. Enteric coatings are well-known in the art. For example, enteric coatings are available under the trade name Eudragit™. In another embodiment, the particles can be encapsulated in an enteric capsule, wherein the enteric polymer is a component of the capsule shell.

The particles can be dispersed in a bioadhesive matrix. For examples, the particles can be dispersed in a bulk polymer, which itself is bioadhesive. The resulting dispersion can be encapsulated in a capsule, such as a hard or soft gelatin or non-gelatin capsule or formulated as a tablet or caplet. The dosage form can be coated to modify release of the agent as described above.

### II. Methods of Making Particles

The bioactive agent may be micronized prior to incorporation into the microparticles or nanoparticles of agent. In some embodiments, the micronization is cryo-emulsion micronization.

Optionally, the micro- or nanoparticles containing the bioactive agent include one or more biocompatible polymers, such as the biocompatible polymers described above. The identity and quantity of the one or more additional polymers can be selected, for example, to influence particle stability, i.e. that time required for distribution to the site where delivery is desired, and the time desired for delivery. Pharmaceutically acceptable excipients, including pH modifying agents, disintegrants, preservatives, and antioxidants, can be incorporated during micro- and nanoparticle formation.

Common microencapsulation techniques include, but are not limited to, spray drying, interfacial polymerization, phase separation encapsulation (spontaneous emulsion microencapsulation, solvent evaporation microencapsulation, and solvent removal microencapsulation), coacervation, low temperature microsphere formation, and phase inversion nanoencapsulation (PIN).

The compositions can include a matrix and with the bioactive agent entrapped, entrained or otherwise associated with the matrix and can be formed as described in published PCT application No. WO 2016/025911 to Brown University and TherapyX, Inc. The disclosure of which is incorporated herein.

Exemplary methods of micro- and nanoparticle formulation are briefly described below.

A. Spray Drying

Spray drying could be used to make micronized proteins as well as encapsulated proteins after micronization. Methods for forming encapsulated microspheres/nanospheres using spray drying techniques are described in U.S. Pat. No. 6,620,617, to Mathiowitz et al. In this method, the polymer, optionally with one or more excipients, is dissolved in an organic solvent such as methylene chloride or in water. Alternative solvent systems are known, and include a mixture of water and tert-butyl alcohol (TBA). A known amount of one or more active agents to be incorporated in the particles is suspended (in the case of an insoluble active agent) or co-dissolved (in the case of a soluble active agent) in the polymer solution. Preferably, the active agent and the polymer dissolve in the solvent system.

The solution or dispersion is pumped through a micronizing nozzle driven by a flow of compressed gas, and the resulting aerosol is suspended in a heated cyclone of air, allowing the solvent to evaporate from the microdroplets, forming particles. Microspheres/nanospheres ranging between 0.1-10 microns can be obtained using this method. Preferably the particles formed by this spray drying step range from about 1 to about 10 μm in size

B. Phase Separation Microencapsulation

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

1. Spontaneous Emulsion Microencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

2. Solvent Evaporation Microencapsulation

Methods for forming microspheres using solvent evaporation techniques are described in E. Mathiowitz et al., J. Scanning Microscopy, 4:329 (1990); L. R. Beck et al., Fertil. Steril., 31:545 (1979); L. R. Beck et al Am J Obstet Gynecol 135(3) (1979); S. Benita et al., J. Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microspheres/nanospheres. This method is useful for relatively stable polymers like polyesters and polystyrene. However, labile polymers, such as polyanhydrides, may degrade during the fabrication process due to the presence of water. For these polymers, some of the following methods performed in completely anhydrous organic solvents are more useful.

3. Solvent Removal Microencapsulation

The solvent removal microencapsulation technique is primarily designed for polyanhydrides and is described, for example, in WO 93/21906 to Brown University Research Foundation. In this method, the substance to be incorporated is dispersed or dissolved in a solution of the selected polymer in a volatile organic solvent, such as methylene chloride. This mixture is suspended by stirring in an organic oil, such as silicon oil, to form an emulsion. Microspheres that range between 1-300 microns can be obtained by this procedure. Substances which can be incorporated in the microspheres include pharmaceuticals, pesticides, nutrients, imaging agents, and metal compounds.

C. Coacervation

Encapsulation procedures for various substances using coacervation techniques are known in the art, for example, in GB-B-929 406; GB-B-929 40 1; and U.S. Pat. Nos. 3,266,987, 4,794,000, and 4,460,563. Coacervation involves the separation of a macromolecular solution into two immiscible liquid phases. One phase is a dense coacervate phase, which contains a high concentration of the polymer encapsulant (and optionally one or more active agents), while the second phase contains a low concentration of the polymer. Within the dense coacervate phase, the polymer encapsulant forms nanoscale or microscale droplets. Coacervation may be induced by a temperature change, addition of a non-solvent or addition of a micro-salt (simple coacervation), or by the addition of another polymer thereby forming an interpolymer complex (complex coacervation).

D. Low Temperature Casting of Microspheres

Methods for very low temperature casting of controlled release microspheres are described in U.S. Pat. No. 5,019,400 to Gombotz et al. In this method, a polymer is dissolved in a solvent optionally with one or more dissolved or dispersed active agents. The mixture is then atomized into a vessel containing a liquid non-solvent at a temperature below the freezing point of the polymer-substance solution which freezes the polymer droplets. As the droplets and non-solvent for the polymer are warmed, the solvent in the droplets thaws and is extracted into the non-solvent, resulting in the hardening of the microspheres.

E. Phase Inversion Nanoencapsulation (PIN)

Particles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non-solvent for the polymer, to spontaneously produce, under favorable conditions, polymeric microspheres, wherein the polymer is either coated with the particles or the particles are dispersed in the polymer. See, e.g., U.S. Pat. No. 6,143,211 to Mathiowitz, et al. The method can be used to produce monodisperse populations of nanoparticles and microparticles in a wide range of sizes, including, for example, about 100 nanometers to about 10 microns.

Advantageously, an emulsion need not be formed prior to precipitation. The process can be used to form microspheres from thermoplastic polymers.

F. Sequential Phase Inversion Nanoencapsulation (sPIN)

Multi-walled nanoparticles can also be formed by a process referred to as “sequential phase inversion nanoencapsulation” (sPIN), which is described in U.S. Pat. No. 8,673,359 to Cho, et al. sPIN is particularly suited for forming monodisperse populations of nanoparticles, avoiding the need for an additional separations step to achieve a monodisperse population of nanoparticles.

### III. Methods of Use

A. Methods of Treatment

The compositions can be formulated into a variety of different drug delivery dosage forms and administered to a patient by any suitable method, including oral, injection (subcutaneous, intramuscular, intravenous), sublingual, inhalation, and transdermal delivery. Most typically, the compositions are formulated for and/or delivered by oral administration.

The compositions can be administered in an effective amount to a subject in need thereof. The terms “effective amount” and “therapeutically effective amount” typically means a dosage sufficient to alleviate one or more symptoms of a disorder, disease, or condition being treated, or to otherwise provide a desired pharmacologic and/or physiologic effect. In some embodiments, the composition leads to increased bioavailability, bioactivity, or a combination thereof an active agent relative to a control. In some embodiments, the composition allows the active agent to be used at a lower dosage or less frequent administrations relative to a control.

GLP-1 and analogues thereof can be administered to significantly reduce fasting blood glucose, post-prandial blood glucose, HbA1c, weight, and/or daily insulin requirements (see Gupta, Indian J Endocrinol Metab. 2013 May-June; 17(3): 413-421.). In some embodiments, the composition is administered to reduce fasting blood glucose, post-prandial blood glucose, glycated haemoglobin (HbA1c), weight, or daily insulin requirements, or a combination thereof.

GLP-1 analogues also can be administered to treat Type I and Type II diabetes, and have shown a substantial beneficial pleiotropic effect, extending to virtually every organ system. For example, GLP-1 analogues have been shown improve cardiovascular parameters, having a positive effect on myocardial contractility, hypertension (natriuretic/diuretic effect), endothelium (anti-atherosclerotic), and lipid profile (improvement in HDL cholesterol, fasting triglycerides).

GLP-1 analogues can be administered to facilitate neuronal protection, resulting in an improvement in cognition, memory, and spatial learning. It modifies eating behavior by inducing satiety, thereby reducing energy intake by approximately 12%. Via interaction with the peripheral nervous system (vagus) central, GLP-1 augmentation causes gastric slowing, inducing a post-prandial satiety. Weight loss, which can also be induced by GLP-1 analogues, is dose dependent and progressive.

GLP-1 can be administered to reduce insulin sensitivity through restoration of insulin signaling and reduction of hepatic gluconeogenesis. Enhanced insulin secretion causes increased uptake of glucose in the muscle and adipocytes, and reduced expression of glucose from the liver. By promoting weight loss, GLP-1 analogues can improve peripheral insulin-mediated glucose uptake. Reduced insulin resistance is evident locally, at the level of beta-cell and fat cell (reduced release of free fatty acids) and systemically (down-gradation of markers of inflammation).

Thus, in some embodiments the compositions are administered in an effective amount to improve cardiovascular heath, enhance neuroprotection, induce weight loss, reduce insulin sensitivity, or a combination thereof. In some embodiments, the compositions are administered in an effect amount to alter one or more physiological or biochemical parameters or symptoms discussed herein.

The precise dosage of compositions will vary according to a variety of factors such as subject-dependent variables (e.g., age, immune system health, etc.), the disease or disorder being treated, as well as the route of administration and the pharmacokinetics of the agent being administered. Exemplary dosages for subcutaneous administration of common GLP-1 analogues are introduced above and otherwise known in the art. In some embodiments, the dosage of GLP-1 or an analogue thereof administered in the disclosed particle formulations is the same or similar to those mentioned above or known in the art. In some embodiments, the dosage is higher or lower that the art recognized dosage. For example, in some embodiments, the dosage GLP-1 or analogue thereof in a particle formulation administered orally is the same or higher than a traditional subcutaneous administration without particles. In some embodiments, the dosage GLP-1 or analogue thereof in a particle formulation administered subcutaneously is the same or lower than a traditional subcutaneous administration without particles.

B. Combination Therapy

The disclosed compositions can be used alone, or in combination with one or more additional therapeutic agents. For example, GLP-1 analogues are currently approved for treatment of type II diabetes as a “monotherapy” and as “add-on therapy” to existing anti-diabetes medication (mono, dual, or triple therapy). In some embodiments, the one or more additional therapeutic agents are selected from the group consisting of biguanides, sulfonylureas, meglitinide derivatives, alpha-glucosidase inhibitors, thiazolidinediones (tzds), dipeptidyl peptidase iv (dpp-4) inhibitors, selective sodium-glucose transporter-2 (sglt-2) inhibitors, insulins, amylinomimetics, bile acid sequestrants, and dopamine agonists. The additional therapeutic agent can be in the same or different admixture with the GLP-1 or analogue thereof. The additional therapeutic agent can be administered at the same time or at a different time from the GLP-1 or analogue thereof.

The disclosed polymeric particles, compositions, pharmaceutical compositions, and methods can be further understood through the following numbered paragraphs.

1. A composition comprising Glucagon-like peptide-1 (GLP-1) or an analogue thereof entrapped in or incorporated into polymeric particles that increase the bioactivity, bioavailability, or a combination thereof of the GLP-1 or analogue thereof when orally administered to a subject in need thereof compared to administration of the GLP-1 or analogue thereof alone,

wherein the polymeric particles do not consist of poly(lactide-co-glycolide)-COOH.

2. A composition comprising Glucagon-like peptide-1 (GLP-1) or an analogue thereof entrapped in or incorporated into polymeric particles that increase the bioactivity, bioavailability, or a combination thereof of the GLP-1 or analogue thereof when orally administered to a subject in need thereof compared to administration of the GLP-1 or analogue thereof alone, wherein the bioavailability of the GLP-1 or analogue thereof is at least 40%, at least 45%, at least 50%, or at least 60%,

wherein the bioavailability is determined using an intraperitoneal glucose tolerance test (IPGTT) and comparing serum glucose levels over time for the orally administered composition with serum glucose levels over time for an intraperitoneally injected formulation containing unencapsulated GLP-1 or the analogue thereof at same dosage as the composition.

3. The composition of paragraph 1 or paragraph 2, wherein the polymeric particles comprise one or more biodegradable polyesters or polyanhydrides, or a co-polymer, or blend thereof.

4. A composition comprising Glucagon-like peptide-1 (GLP-1) or an analogue thereof entrapped in or incorporated into polymeric particles comprising one or more biodegradable polyesters or polyanhydrides, or a co-polymer, or blend thereof, wherein the polymeric particles do not consist of poly(lactide-co-glycolide)-COOH.

5. The composition of any one of paragraphs 1-4, wherein the polymeric particles comprise a polymer comprising lactic acid units.

6. The composition of any one of paragraphs 1-5, wherein the polymeric particles comprise polymer comprising adipic acid units.

7. The composition of any one of paragraphs 1-6, wherein the polymeric particles comprise a co-polymer comprising two or more biodegradable polyesters or polyanhydrides.

8. The composition of any one of paragraphs 1-7, wherein the polymeric particles comprise a blend of polymers comprising two or more biodegradable polyesters or polyanhydrides.

9. The composition of any one of paragraphs 1-8, wherein the polymeric particles comprise poly(adipic acid), poly-L-lactic acid, poly-D-lactic acid, poly-D,L-lactic acid, poly-L-lactide, poly-D-lactide, poly-D,L-lactide, poly(lactide-co-glycolide), or copolymer or blend thereof.

10. The composition of any one of paragraphs 1-9, wherein the polymeric particles comprise a blend of poly-L-lactic acid, poly-D-lactic acid, poly-D,L-lactic acid, poly-L-lactide, poly-D-lactide, or poly-D,L-lactide and poly(lactide-co-glycolide).

11. The composition of any one of paragraphs 1-10, wherein the polymeric particles are microparticles or nanoparticles.

12. The composition of any one of paragraphs 1-11, wherein the polymeric particles are formed by Phase Inversion Nanoencapsulation (PIN).

13. The composition of any one of paragraphs 1-12, comprising GLP-1.

14. The composition of any one of paragraphs 1-12, comprising an analogue of GLP-1.

15. The composition of paragraph 14, wherein the analogue of GLP-1 is selected from the group consisting of exenatide, liraglutide, lixisenatide, albiglutide, dulaglutide, semaglutide, and taspoglutide.

16. A pharmaceutical composition comprising the composition of any one of paragraphs 1-15 and a pharmaceutically acceptable carrier.

17. The pharmaceutical composition of paragraph 16, wherein the pharmaceutical composition is formulated for oral administration.

18. Use of the composition of any one of paragraphs 1-15 or the pharmaceutical composition of any one of paragraphs 16-17 in a mammalian subject to reduce fasting blood glucose, post-prandial blood glucose, glycated haemoglobin (HbA1c), weight, daily insulin requirements, or a combination thereof.

19. The use of paragraph 18, wherein the mammalian subject is a human.

20. The use of paragraph 19 wherein the mammalian subject has type I or type II diabetes mellitus.

21. Use of the composition of any one of paragraphs 1-15 or the pharmaceutical composition of any one of paragraphs 16-17 for treating type II diabetes mellitus in a mammalian subject.

22. Use of the composition of any one of paragraphs 1-15 or the pharmaceutical composition of any one of paragraphs 16-17 to improve the cardiovascular condition of a mammalian subject, such as by improving the subject's myocardial contractility, hypertension, endothelium, lipid profile, or a combination thereof.

23. Use of the composition of any one of paragraphs 1-15 or the pharmaceutical composition of any one of paragraphs 16-17 to improve cognition, memory, spatial learning, or a combination thereof in a mammalian subject.

24. Use of any one of paragraphs 30 to 36, wherein the composition or the pharmaceutical composition is suitable for oral delivery.

25. A method of treating a mammalian subject in need thereof comprising administering to the mammalian subject an effective amount of the composition of any one of paragraphs 1-15 or the pharmaceutical composition of any one of paragraphs 16-17 to reduce fasting blood glucose, post-prandial blood glucose, glycated haemoglobin (HbA1c), weight, daily insulin requirements, or a combination thereof.

26. The method of paragraph 25, wherein the mammalian subject has type I or type II diabetes mellitus.

27. A method of treating type II diabetes mellitus comprising administering a mammalian subject an effective amount of the composition of any one of paragraphs 1-15 or the pharmaceutical composition of any one of paragraphs 16-17.

28. A method of improving the cardiovascular condition of a mammalian subject in need thereof comprising administering to the subject an effective amount of the composition of any one of paragraphs 1-15 or the pharmaceutical composition of any one of paragraphs 16-17 to improve myocardial contractility, hypertension, endothelium, lipid profile, or a combination thereof.

29. The method of treating a mammalian subject in need thereof comprising administering to the subject an effective amount of the of any one of paragraphs 1-15 or the pharmaceutical composition of any one of paragraphs 16-17 to improve cognition, memory, spatial learning, or a combination thereof in the subject.

30. The method of any one of paragraphs 18-22, where in the composition or the pharmaceutical composition is orally administered to the subject.

Those skilled in the art will recognize, or be able to ascertain using no more than routine experimentation, many equivalents to the specific embodiments of the invention described herein. Such equivalents are intended to be encompassed by the ensuing claims.

The present invention will be further understood by reference to the following non-limiting examples.

### EXAMPLES

### Example 1

### GLP-1 that is Encapsulated in Nanoparticles is Released in a Sustained Manner and in an Active Form In Vitro

### Materials and Methods

Nanoparticle Formulations

GLP-1 (7-36 amide) was encapsulated into different nanoparticle formulations and the formulations were analyzed for release kinetics. Three different base polymers were investigated for use in encapsulating GLP-1. The polymers were; PAA (poly-adipic acid), PLGA (poly-lactic-co-glycolic acid) and PLA (poly-lactic acid). These polymers have been studied for use as biodegradable delivery systems.

Nanoparticle Preparation

The preparation of nanoparticles using the PIN method has been described previously in E. Mathiowitz, et al., “Biologically Erodable Microspheres as Potential Oral Drug Delivery Systems,” Nature 386, 410-414, 1997. Briefly, PAA, PLA or PLGA, (Birmingham Polymers, Inc., Birmingham, Ala.) plus recombinant GLP-1 (7-36 amide) (BACHEM Americas, King of Prussia, Pa.) in methylene chloride (Fisher, Pittsburgh, Pa.) are rapidly poured into light petroleum ether (Fisher, Pittsburgh, Pa.) for formation of nanoparticles. The particles are filtered and lyophilized overnight for complete removal of solvent and stored at 4° C. The final GLP-1 loading for all nanoparticle formulations was 2.5% unless otherwise stated.

Quantification of In Vitro GLP-1 (7-36 Amide) Release

The quantity of released GLP-1 was determined using an ELISA kit specific for total GLP-1 (Phoenix Pharmaceuticals, Inc, Belmont, Calif.) as per supplier's instructions. The particles were suspended at 10 mg per ml in buffer consisting of DMEM/F12 10% heat inactivated FCS, 200 mM HEPES and 1× penicillin/streptomycin plus L-glutamine (Gibco, Grand Island, N.Y.). DPP-IV inhibitor (Linco/Millipore, St. Charles, Mo.) was added to 50 uM in each well. Aliquots (200 ul) were placed in triplicate wells for each sample for each time point. The plate was incubated at 37° C. under 5% CO₂ and the medium was collected from separate wells hourly for up to 8 hours, and the samples were stored at −20° C.

Preliminary Stability Study

PIN encapsulated GLP-1 particles were prepared using 3 different polymers. Both total peptide and bioactive peptide (using a second ELISA kit, specific for the complete and active form of GLP-1, Linco/Millipore, St. Charles, Mo.) were quantified in a 3-hour release assay after storage at −70° C. for 2 weeks.

Results

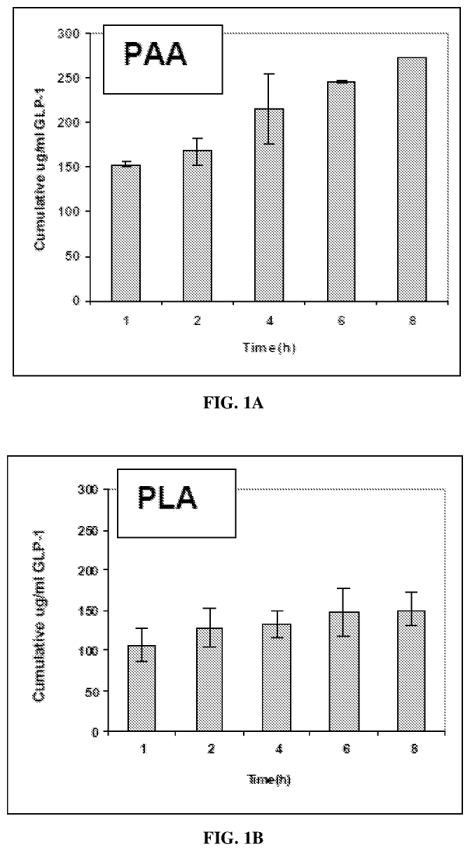

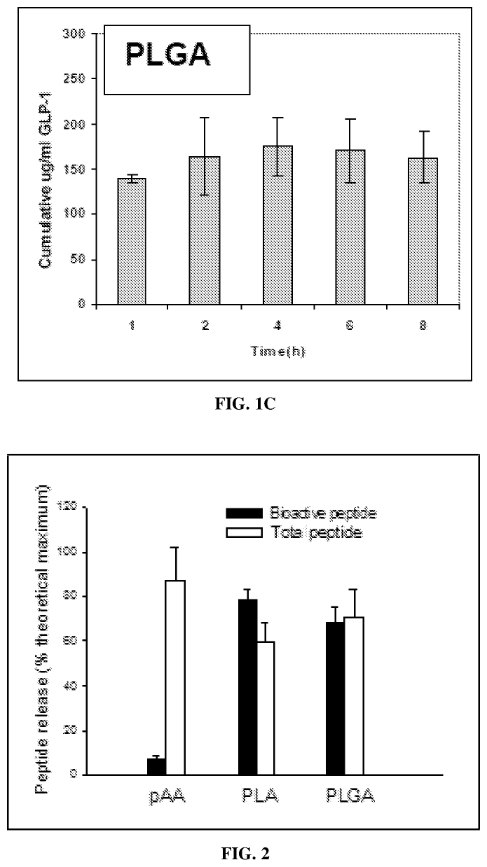

In vitro release profiles of total GLP-1 for each of the three formulations is shown in FIGS. 1A-1C. GLP-1 encapsulated PAA, PLA, or PLGA nanoparticles (10 mg) were hydrated in 0.2 ml media and analyzed for release kinetics as described above. Cumulative release was determined at the indicated times in triplicate. All formulations had a GLP-1 loading of 2.5%.

The release profiles of PAA and PLA display an initial burst followed by continuous release for 6 to 8 hours. In contrast, PLGA exhibited high burst and short-term release (2-4 hours).

Storage stability of each formulation was investigated. The formulations were stored at −70° C. for 2 weeks, then tested for release as described above. The results shown in FIG. 2 demonstrate that only 6.9% of the theoretical maximum for active peptide was detected versus 84% for total peptide in the case of PAA. In contrast PLA demonstrated 77% bioactive vs 62% total peptide released and PLGA showed 68% bioactive vs 70% total peptide released following storage.

The above data demonstrate that peptide release profiles and stability characteristics of the three formulations exhibited distinct differences. PLA and PAA nanoparticle formulations provided longer-term in vitro release of GLP-1 in comparison to PLGA. On the other hand, the PAA-based formulation was found to be highly unstable following two weeks of storage, whereas PLA and PLGA remained fully active.

### Example 2

### GLP-1 Nanoparticles are Biologically Active In Vivo

### Materials and Methods

Formulation Evaluation in an IPGTT Using BKS. Cg WT Mice

The three different GLP-1 nanoparticle formulations constructed with PAA, PLA and PLGA polymers were assessed in an intraperitoneal glucose tolerance test. Each formulation was administered to four mice. An IPGTT was undertaken to avoid the contribution of endogenous incretins that might occur after the oral administration of a meal. Mice were fed orally with GLP-1-loaded PAA, PLA or PLGA nanoparticles hydrated in 0.1 ml of dH₂O using a 22 gauge closed end lacrimal feeding cannula attached to a 1 ml syringe. Mice in control groups received soluble GLP-1 mixed with blank nanoparticles orally. Mice were given an intraperitoneal (i.p.) injection of glucose (3 mg/g body weight in 0.2 ml saline) immediately after the experimental and control nanoparticles were fed to the appropriate mice. Blood was collected from mice before the glucose injection and then at times 15, 45, and 60 minutes after glucose administration. Blood glucose was determined through the use of a blood glucose monitor (Hypoguard Advance Microdraw Blood Glucose Meter).

Bioavailability

BKS.Cg WT mice were treated either with oral PIN/PLA GLP-1 microspheres (10 mg particles containing 250 μg peptide), with an intraperitoneal injection of soluble peptide (250 μg) or oral blank microspheres (glucose only control). All mice were then injected intraperitoneally with glucose (3 mg/ml in 0.2 ml saline). Blood was collected at time 0 (pre-glucose) and at indicated times post-glucose. Serum glucose levels were determined as described above.

Serum Pharmacokinetics

BKS.Cg WT mice were fed 10 mg PLA nanoparticles containing 250 ug GLP-1 or blank control PLA nanoparticles at time 0. Blood was collected at the indicated time points in heparinized tubes and placed into 1.5 ml eppendorf tubes containing 50 ul of 10 mM DPP-IV inhibitor. Serum GLP-1 was determined using the ELISA specific for bioactive peptide.

Results

An experiment was designed to determine whether nanoparticles are able to deliver physiologically relevant amounts of GLP-1 (7-36 amide) to the blood stream of healthy experimental animals, and to identify the formulation with the optimal in vivo efficacy profile. To this end, female BKS.Cg WT mice were treated with a single oral administration of GLP-1 nanoparticle formulations in an IPGTT assay. Pre- and post-treatment blood glucose levels were monitored in experimental and control mice.

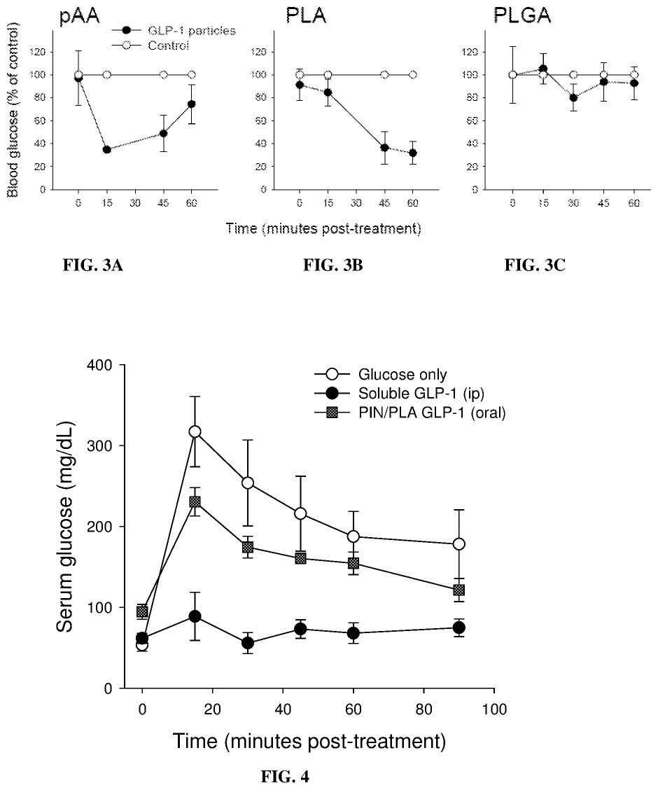

The results are shown in FIGS. 3A-3C. The PAA/GLP-1 particles showed a high amount of activity early (15 minutes) with decreasing activity thereafter (maintaining a 50-60% reduction in blood glucose between 15 and 45 minute time points). PLA particles displayed slower kinetics, were equally active (60-65% reduction in blood glucose levels) yet were effective longer (for at least 60 minutes). In contrast, PLGA/GLP-1 nanoparticles did not achieve a significant reduction in blood glucose and were not effective long-term.

According to the foregoing assays, PIN/PLA particles provided the best combination of release rate, stability and in vivo efficacy characteristics. The ability of oral PIN/PLA GLP-1 particles to reduce blood glucose was compared to that of intraperitoneally injected free peptide in an IPGTT. Three different concentrations of GLP-1 (30, 90 and 250 ug/mouse) were evaluated.

The results from the 250 μg GLP-1 study are shown in FIG. 4. The oral formulation reduced blood glucose in the mice significantly, however the short-term efficacy of intraperitoneally soluble peptide was superior. Similar results were obtained with 30 and 90 μg of peptide. Area under the curve (AUC) analysis demonstrated that the relative bioavailability for the oral formulation was between 43-51% depending on the dose (Table 1). These data demonstrate that oral administration of PIN/PLA GLP-1 resulted in effective peptide delivery to the blood.

TABLE 1
Area under the curve and relative bioavailability study for
PIN/PLA GLP-1 particles*.

| AUC_{30 ug} | | AUC_{90 ug} | | AUC_{250 ug} | |
|---|---|---|---|---|---|
| Free | Encap | Free | Encap | Free | Encap |
| 7515 | 16050 | 7425 | 14827.5 | 6262 | 14445 |

| | | | |
|---|---|---|---|
| Relative | 46.8 | 50.1 | 43.4 |
| Bioavailability (%) | | | |

$* AUC was calculated using the formula \text{:} \frac{[ AUC ] po / dose po}{[ AUC ] i . p . / dose i . p .} \times 100$

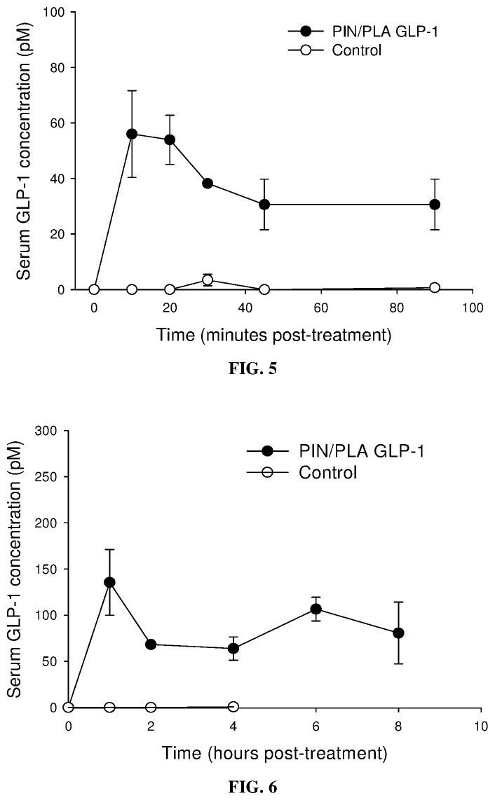

Serum GLP-1 was determined using the ELISA specific for bioactive peptide. The results for short- and long-term release are shown in FIGS. 5 and 6, respectively.

The above data demonstrates that mice receiving PLA/GLP-1 nanoparticles maintained serum GLP-1 levels at or above 25 pM for at least 90 minutes. In contrast, control mice that received blank nanoparticles did not display significant serum GLP-1 levels at any time point measured.

These data show that a single oral administration of GLP-1 PIN/PLA particles can sustain physiologically relevant serum levels of bioactive GLP-1 for at least 8 hours. The absolute levels of GLP-1 displayed in FIG. 6 were higher than those observed in the short-term study (FIG. 5) as the mice used in the second study were significantly smaller (˜25 g vs ˜17 g in the short-term vs long-term study, respectively).

### Example 3

### Orally Delivered GLP-1 Nanoparticles are Effective in Restoring Normo-Glycemia in Lepr^{db/db} Mice

Materials and Methods

Efficacy of GLP-1 Nanoparticle Treatment

To determine the efficacy of GLP-1 nanoparticle treatment on the fasting glucose levels in a diabetic mouse model, Lepr^{db/db} mice were fasted overnight to minimize potential variability in particle absorption that can be caused by the presence of food in the digestive tract. Eight to 10 week old diabetic mice were pre-bled to establish a baseline blood glucose level. At time 0 the PIN/PLA GLP-1 group received 30 mg of PIN/PLA GLP-1 particles (600 ug peptide at 2% loading). Four mice were tested in the soluble peptide group, and received 600 ug of free GLP-1 mixed with blank particles. The control group (four mice) received 30 mg blank PIN/PLA particles. The ability of the GLP-1 nanoparticles to lower blood glucose was then compared to that of orally delivered soluble GLP-1 (7-36 amide) mixed with blank nanoparticles or blank nanoparticle only controls. Blood glucose levels were assayed at given times after particle administration.

Results

This experiment was designed to determine whether GLP-1 PIN/PLA particles would be effective in controlling the fasting blood glucose levels in diabetic Lepr^{db/db} mice. Like an untreated diabetic patient the Lepr^{db/db} mouse exhibits a constitutively high level of blood glucose and is regularly used as an animal model for Type 2 Diabetes. Lepr^{db/db} mice manifest insulin resistance at 2 weeks and develop hyperglycemia at around 7 weeks of age.

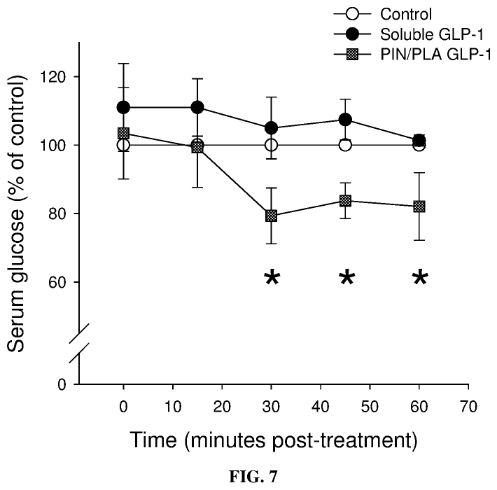

The results displayed in FIG. 7 show that oral administration of GLP-1/PLA nanoparticles reduced blood glucose in Lepr^{db/db} mice with established disease. Blood glucose values were significantly lower than controls at 30 (p=0.004), 45 (p=0.002) and 60 (p=0.005) minutes. Oral delivery of free GLP-1 peptide mixed with blank PLA particles had no significant effect on blood glucose in this test.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. (canceled)

2. A composition comprising Glucagon-like peptide-1 (GLP-1) or an analogue thereof entrapped in or incorporated into polymeric particles that increase the bioactivity, bioavailability, or a combination thereof of the GLP-1 or analogue thereof when orally administered to a subject in need thereof compared to administration of the GLP-1 or analogue thereof alone, wherein the bioavailability of the GLP-1 or analogue thereof is at least 40%, at least 45%, at least 50%, or at least 60%,
wherein the bioavailability is determined using an intraperitoneal glucose tolerance test (IPGTT) and comparing serum glucose levels over time for the orally administered composition with serum glucose levels over time for an intraperitoneally injected formulation containing unencapsulated GLP-1 or the analogue thereof at same dosage as the composition.

3. (canceled)

4. The composition of claim 2, wherein the polymeric particles comprise one or more biodegradable polyesters or polyanhydrides, or a co-polymer, or blend thereof.

5. (canceled)

6. The composition of claim 1, wherein the polymeric particles comprise a polymer comprising lactic acid units.

7. The composition of claim 1, wherein the polymeric particles comprise polymer comprising adipic acid units.

8. The composition of claim 1, wherein the polymeric particles comprise a co-polymer comprising two or more biodegradable polyesters or polyanhydrides.

9. The composition of claim 1, wherein the polymeric particles comprise a blend of polymers comprising two or more biodegradable polyesters or polyanhydrides.

10. The composition of claim 1, wherein the polymeric particles comprise poly(adipic acid), poly-L-lactic acid, poly-D-lactic acid, poly-D,L-lactic acid, poly-L-lactide, poly-D-lactide, poly-D,L-lactide, poly(lactide-co-glycolide), or copolymer or blend thereof.

11-13. (canceled)

14. The composition of claim 12, comprising GLP-1.

15. The composition of claim 12, comprising an analogue of GLP-1.

16. The composition of claim 15, wherein the analogue of GLP-1 is selected from the group consisting of exenatide, liraglutide, lixisenatide, albiglutide, dulaglutide, semaglutide, and taspoglutide.

17. A pharmaceutical composition comprising
a composition comprising Glucagon-like peptide-1 (GLP-1) or an analogue thereof entrapped in or incorporated into polymeric particles that increase the bioactivity, bioavailability, or a combination thereof of the GLP-1 or analogue thereof when orally administered to a subject in need thereof compared to administration of the GLP-1 or analogue thereof alone, wherein the bioavailability of the GLP-1 or analogue thereof is at least 40%, at least 45%, at least 50%, or at least 60%,
wherein the bioavailability is determined using an intraperitoneal glucose tolerance test (IPGTT) and comparing serum glucose levels over time for the orally administered composition with serum glucose levels over time for an intraperitoneally injected formulation containing unencapsulated GLP-1 or the analogue thereof at same dosage as the composition and
a pharmaceutically acceptable carrier.

18-28. (canceled)

29. The pharmaceutical composition of claim 17, wherein the pharmaceutical composition is formulated for oral administration.

30. A method of treating a mammalian subject in need thereof, comprising, administering to the mammalian subject
a composition comprising Glucagon-like peptide-1 (GLP-1) or an analogue thereof entrapped in or incorporated into polymeric particles that increase the bioactivity, bioavailability, or a combination thereof of the GLP-1 or analogue thereof when orally administered to a subject in need thereof compared to administration of the GLP-1 or analogue thereof alone, wherein the bioavailability of the GLP-1 or analogue thereof is at least 40%, at least 45%, at least 50%, or at least 60%,
wherein the bioavailability is determined using an intraperitoneal glucose tolerance test (IPGTT) and comparing serum glucose levels over time for the orally administered composition with serum glucose levels over time for an intraperitoneally injected formulation containing unencapsulated GLP-1 or the analogue thereof at same dosage as the composition.

31. The method of claim 30, wherein the mammalian subject is a human.

32. The method of claim 31 wherein the mammalian subject has type I or type II diabetes mellitus.

33. The method of claim 32, wherein the administering step comprises administering an effective amount of the composition to treat one or more symptoms associated with type II diabetes mellitus in the mammalian subject.

34. The method of claim 31, wherein the administering step comprises administering an effective amount of the composition to improve the cardiovascular condition of the mammalian subject, such as by improving the subject's myocardial contractility, hypertension, endothelium, lipid profile, or a combination thereof.

35. The method of claim 31, wherein the administering step comprises administering an effective amount of the composition to improve cognition, memory, spatial learning, or a combination thereof in the mammalian subject.

36. The method of claim 31, wherein the administering step comprises orally administering of the composition.

37. The method of claim 31, wherein the administering step comprises administering an effective amount of the composition to reduce fasting blood glucose, post-prandial blood glucose, glycated haemoglobin (HbA1c), weight, daily insulin requirements, or a combination thereof in the mammalian subject.
